(19) United States
(12) Patent Application Publication
(10) Pub. No.: US 2016/0345921 A1
(43) Pub. Date: Dec. 1, 2016

(54) Device and Method for Multimodal Imaging

(71) Applicant: Berthold Technologies GmbH & Co. KG, Bad Wildbad (DE)

(72) Inventors: Frank SCHLEIFENBAUM, Birkenfeld (DE); Fritz BERTHOLD, Pforzheim (DE)

(21) Appl. No.: 15/166,383

(22) Filed: May 27, 2016

(30) Foreign Application Priority Data
May 29, 2015 (DE) 10 2015 209 954.7

(51) Int. Cl. A61B 6/00; A61B 6/08; G21K 1/06

(52) U.S. Cl. CPC A61B 6/4417; A61B 6/508; A61B 6/4233; G21K 1/062; A61B 6/08

(57) ABSTRACT

A device for a multimodal imaging examination of an object, in particular for a multimodal imaging examination of a living object, includes an object carrier for accommodating at least one object to be examined, an x-ray radiation source for emitting x-ray radiation, a first partial system for generating a first image of the object in a first image recording mode and a second partial system for generating a second image of the object in a second image recording mode. The first partial system has a direct digital x-ray detector which is configured to convert x-ray radiation modified by the object into a digital image of the object. The second partial system has a camera system which is configured to convert radiation in the visible wavelength range or UV wavelength range emanating from an object into a digital image of the object.

### CROSS REFERENCE TO RELATED APPLICATION

This application claims priority under 35 U.S.C. §119 from German Patent Application No. 10 2015 209 954.7, filed May 29, 2015, the entire disclosure of which is herein expressly incorporated by reference.

### BACKGROUND AND SUMMARY OF THE INVENTION

The invention relates to a device and a method for a multimodal imaging examination of an object. A preferred field of application is the multimodal imaging examination of a living object.

In recent years, the use of small animals, in particular rodents, as a model system has become very important in preclinical trials. Methods which render it possible to study disease and progress of disease over a relatively long time on living objects are standard methods in modern pharmacology and tumour research. Here, the examination of complete living objects is superior to the study of isolated cell systems since many or all physiological factors of interest, such as e.g. neuronal, hormonal, nutritional or immunological influences can be inherently taken into account and examined in the complete physiological context. Furthermore, the examination of small animals permits systemic studies which, for example, examine the formation and spatial distribution of metastases. Imaging methods are particularly well-suited to a non-invasive examination of living animals, ideally over a relatively long period of time. Here, the object to be examined remains uninfluenced to the greatest possible extent by the examination; it is possible to dispense with the removal of tissue material.

When small animal research started out in the preclinical field, use was made of instruments for human medicine which were designed for the clinical examination of human patients. However, dedicated instruments designed only for the examination of small animals, in particular mice and rats, were quickly established for practical and regulatory reasons. Specialized small animal imaging systems were adapted to the special requirements of small animal research and initially had the same imaging modalities as the clinical systems. At first, use was made, in particular, of x-ray methods (2D and computer tomography) and nuclear magnetic resonance methods.

However, these methods only supply morphological information and can hardly provide functional statements. The tracking of cell growth or the tracking of the expression of defined target proteins as these develop over time is of great importance for tumour research in particular, but also for pharmaceutical development. Optical methods using fluorescence and luminescence are generally suitable methods for imaging these processes. While these methods cannot be used in humans due to the limited penetration depth, they are suitable for small animal imaging since emissions emanating from organs in the interior can also be recorded due to the small body dimensions.

Modern approaches in small animal imaging attempt to further increase the information content of an image by virtue of combining the information from a plurality of imaging methods (e.g. reflected light photography, luminescence/fluorescence, x-rays) This is typically carried out by the superposition of images which were obtained in different image recording modes. By way of example, the position of a tumour or of metastases can be correlated with the bony skeleton of the experimental animal by such “multiplexing” of image information and hence these can be assigned to a specific organ with greater reliability.

Furthermore, vascular regions can be identified and the perfusion of these regions can be quantified (angiography) and correlated with the tumour growth by way of contrast-enhancing methods, in which a contrast agent which effectively absorbs x-rays is injected into the object (trial animal).

While photographic reflected-light recordings, luminescence recordings and fluorescence recordings can, in principle, be recorded by the same highly sensitive camera system and, in principle, only differ by the type of light generation (e.g. biological, chemical or optical by external irradiation) and optional light filtering (e.g. no filtering in the case of standard luminescence and reflected light, emission filtering in the case of BRET luminescence, excitation and emission filtering in the case of fluorescence), x-ray radiation cannot be recorded directly by an optical camera.

The patent U.S. Pat. No. 7,734,325 B2 discloses a system for a multimodal imaging examination of an object. The system has a camera system for recording an image of the object by means of fluorescence, luminescence or bright field photography in one image recording mode. In order to generate an x-ray image of the same small animal, a movable phosphor screen is pushed between the small animal and the optical camera system in another image recording mode and an x-ray source is switched on. X-rays which pass through the small animal are incident on the phosphor screen, in which spatially resolved light emission is generated by scintillation. This light is then detected by the optical camera system by virtue of the fluorescent screen being imaged on the detector area of the camera system. There is no need to move the small animal in this procedure. The same camera is used for both image recording modes. As a result of this, a positionally correct superposition of the images recorded in the different image recording modes is easily possible.

### Problem and Solution

It is an object of the invention to provide a device and a method for a multimodal imaging examination of an object, both of which are suited particularly well to the examination of living objects with a high spatial resolution.

In order to achieve this object, the invention provides a device for a multimodal imaging examination of an object, preferably for a multimodal imaging examination of a living object, comprising: an object carrier for accommodating at least one object to be examined; an x-ray radiation source for emitting x-ray radiation; a first partial system for generating a first image of the object in a first image recording mode, wherein the first partial system has a direct digital x-ray detector which is configured to convert x-ray radiation modified by the object into a digital image of the object; a second partial system for generating a second image of the object in a second image recording mode, wherein the second partial system has a camera system which is configured to convert radiation in the visible wavelength range or UV wavelength range emanating from an object into a digital image of the object. To further achieve this object, a method is provided for a multimodal imaging examination of an object, preferably for a multimodal imaging examination of a living object, comprising the following steps: accommodating the object on an object carrier; irradiating the object with x-ray radiation from an x-ray radiation source; generating a first image of the object in a first image recording mode by means of a direct digital x-ray detector which converts x-ray radiation modified by the object into a digital image of the object; generating a second image of the object in a second image recording mode by means of a camera system which converts radiation in the visible wavelength range or UV wavelength range emanating from the object into a digital image of the object; evaluating the first image and the second image.

The inventors identified that conventional methods which operate with a phosphor screen (fluorescent screen) that is optionally movable into the beam path have disadvantages due to the principles thereof, inter alia in view of the achievable spatial resolution.

Initially, the fluorescent screen should have a sufficient thickness along which the scintillation light is generated, said thickness being in the region of several millimetres for a sufficiently high conversion efficiency. Since said scintillation light is imaged by way of an optical system on the camera sensor, this image is subject to unsharpness as a matter of principle as no defined image plane is present. As a result of this, the achievable spatial resolution is limited and fine structures can only be depicted to limited extent.

Furthermore, it may be the case that the radiation conversion efficiency and the solid angle-restricted light collection efficiency are restricted such that this arrangement has a relatively low sensitivity (detective quantum efficiency, DQE). As a result of this, the obtainable image quality is restricted or high radiation doses are required, which high radiation doses are damaging to the object (e.g. experimental animal) and/or may have an unwanted influence on the tumour growth, especially in the case of tumour studies.

Furthermore, the image recording times can become very long due to the limited sensitivity. As a result, the applicability, for example in the field of angiography, is limited because the flow of the contrast agent can be very high as a result of the high heart rate of small animals, which may be up to 600 beats per minute in the case of mice, and so comparatively high frame rates and short recording times are required for tracking the spread.

These disadvantages can be largely avoided or mitigated if a direct digital x-ray detector is used for image generation in the first image recording mode. A “direct digital x-ray detector” within the meaning of this application is a dedicated x-ray detection unit which is able to convert x-ray radiation modified by the object directly into a digital image of the object without an interposed separate phosphor screen and without interposed imaging optical units, and which therefore directly generates x-ray images in digital form.

In accordance with one development, use is made of a direct digital x-ray detector in the form of a flat panel x-ray detector. Other common names for a flat panel x-ray detector are flat panel detector for x-rays or imaging two-dimensional x-ray detector. A flat panel x-ray detector has a two-dimensionally extended detector area and can therefore generate image information in many area elements at the same time.

As a possibly more cost-effective alternative, it is also possible to use a direct digital x-ray detector which has an x-ray line detector in conjunction with a scanner drive for moving the x-ray line detector across the direction of extent of one line of x-ray radiation-sensitive cells. As a result, a two-dimensional image of a region of interest can be generated successively line-by-line by means of a scanning movement.

Use can be made of different types of direct digital x-ray detectors.

In one embodiment, the direct digital x-ray detector has a photosensitive pixel arrangement which is coated with a scintillator which converts x-ray radiation into visible light. By way of example, the photoactive line or area can have a capacitor, a thin-film transistor and a photodiode for each image point, said photodiode generating a number of electrons proportional to the amount of light generated by the scintillator by way of the inner photoelectric effect. This amount of charge is stored in the capacitor and it can be read out with pixel accuracy by way of the thin-film transistor. Since the scintillator is applied directly onto the photoactive line or area, this results in a large angular range of scintillation light detection, and imaging by way of an optical system is dispensed with and hence the principle-based restrictions accompanying the imaging do not apply.

In some embodiments, a further improvement in the image resolution can be achieved by virtue of use being made of structured scintillators which consist of many individual scintillators delimited from one another. As a result, light scattering is avoided or reduced in comparison with a continuous, unstructured scintillator layer and the scintillation light is mainly guided through the scintillator to the active pixel area (one or a few) assigned thereto and hence the image resolution can be further increased as there is no lateral spatial spread between point of incidence of the x-ray radiation and detection of the light signal.

The digital x-ray detectors operating with scintillators can also be referred to as indirect x-ray detectors since a conversion step for the x-ray radiation is interposed.

Another embodiment uses a so-called direct x-ray detector, which uses a photoconductor sensitive to x-ray radiation instead of an arrangement of scintillator and photodiode structure, said photoconductor generating charges proportional to the amount of incident radiation after the incidence of x-ray photons. By way of example, such a photoconductor can consist of amorphous selenium which has a high absorption cross section for x-ray radiation. Incident x-ray radiation generates electron-hole pairs in the selenium layer, which pairs are separated by an applied external electric field. The electrons move along the electric field and experience substantially no lateral deflection, as a result of which a high spatial resolution can be achieved by way of these layers. The detection of the generated electrons is carried out in an analogous manner by way of storage capacitors and thin field transistors.

Corresponding variants are also possible in the case of a line x-ray detector.

More recent developments use flat panel x-ray detectors based on plastics, in which use is made of a mixture of semiconducting plastics as electron donors and fullerene derivatives as electron acceptors in order to achieve alight-induced charge separation. By way of a simplified production process in which this solution is applied onto a substrate from the liquid phase, it is possible to significantly reduce the production costs. At the same time, these structures achieve a high detection efficiency DQE of up to 75% and in part have better image qualities. Currently, such detectors are still in the testing phase, but it is expected that they will reach a state of market readiness in the foreseeable future. Then, in principle, flat panel detectors based on plastics can be used within the scope of the present invention.

In some embodiments, provision is made for the direct digital x-ray detector to be movably mounted in such a way that the direct digital x-ray detector is movable between a defined first position and at least one defined second position. By way of example, the first position can be an image recording position, in which an x-ray image can be recorded, while the second position is a neutral position, in which no x-ray image can be recorded. Both positions can also respectively be image recording positions.

In some variants, the first position is an image recording position, suitable for recording an x-ray image, for the direct digital x-ray detector, in which the digital x-ray detector is arranged in a detection region of the camera system between the object carrier and the camera system, while the direct digital x-ray detector is arranged outside of the detection region of the camera system in the second position in such a way that an object accommodated on the object carrier is detectable by the camera system. In this case, the system can be converted from a first configuration into a second configuration merely by moving the direct digital x-ray detector, wherein x-ray image recording is possible in the first configuration and one or more images can be recorded by means of the camera system in the second configuration. There is no need to move the object for the change between the two image recording modes; rather, it preferably remains stationary.

Variants in which both the camera system and the direct digital x-ray detector are fixedly installed such that the object is moved from the detection region of the direct digital x-ray detector into the detection region of the camera system, or vice versa, for a change between image recording modes are also possible.

It is possible to arrange the direct digital x-ray detector in a swivellable manner such that it can be folded to-and-fro between an image recording position and a second position not suitable for an image recording. Such a variant can have a space-saving construction.

In some embodiments, the direct digital x-ray detector is fastened to a displacement apparatus in such a way that the direct digital x-ray detector is displaceable in an image recording plane from a first image recording position to at least one laterally offset second image recording position. As a result of this, it is possible, for example, to accommodate a plurality of objects arranged laterally offset and successively record x-ray images of these objects in succession by virtue of the direct digital x-ray detector initially being moved into an image recording position in relation to the first object and being used there for the recording of an image, and thereafter being moved into an image recording position in relation to a next object by way of a lateral displacement and being used there for the recording of an x-ray image, etc.

By way of a linear displacement of the direct digital x-ray detector in an image recording plane with the aid of the displacement apparatus, it is also possible to successively record two or more laterally offset individual images of an individual object which may be larger than the detection area of the direct digital x-ray detector, which individual images can then subsequently be pieced together to form an overall image of the object. As a result of this stitching mode, it is possible to completely detect objects that are possibly many times larger by means of a direct digital x-ray detector with a relatively small detector area.

The use of a line x-ray detector is also realizable with the aid of the displacement apparatus by virtue of said displacement apparatus acting as a scanner apparatus. As mentioned above, these units with substantially the same structure as a flat panel x-ray detector only record the x-ray information in one-dimensional form along one line, as a result of which an image line is obtained for each recording. By way of a linear displacement of the line x-ray detector, for example perpendicular to the direction of extent of a line, it is thus possible to reconstruct a two-dimensional image from a number of individual lines. Here, image recording and image processing correspond, in principle, to the procedure in the stitching mode by means of a flat panel x-ray detector.

A particularly compact configuration of the system can be achieved by virtue of the x-ray radiation source, the object carrier, the direct digital x-ray detector and the camera system being arranged along a common axis, which preferably extends vertically, in one configuration of the system. A change between different operating modes is easily possible by moving the direct digital x-ray detector between an image recording position (between the optical camera system and object carrier apparatus) and a retracted position outside of this region.

There are also embodiments which have a beam splitter which is substantially transparent to x-ray radiation and acts substantially in a reflecting manner for visible light, said beam splitter being arranged or arrangeable in a beam splitter position between the object carrier and the direct digital x-ray detector in such a way that radiation in the visible wavelength range (or UV wavelength range and/or NIR wavelength range) emanating from the object is reflectable by means of the beam splitter in the direction of the camera system and x-ray radiation modified by the object is transmittable in the direction of the direct digital x-ray detector. In this case, the camera system and the direct digital x-ray detector can be installed at fixed positions and it is not necessary to additionally move the object for a change between the illumination modes. In the first image recording mode (x-ray image recording), the x-ray radiation passed by the object and modified by the object can pass through the beam splitter in a substantially unimpeded manner in the direction of the direct digital x-ray detector in order to be converted into a first digital image. By contrast, if the object is illuminated in order to record a reflected light image, a luminescence image or a fluorescence image in the second image recording mode, the portion of the light emanating from the object which reaches the detection region of the camera system is reflected by the wavelength-selectively reflecting beam splitter face of the beam splitter in the direction of the camera system such that at least one second image can be generated.

The images in the first and second image recording mode can be recorded temporally in succession. However, a great advantage of the variant with the beam splitter consists of the fact that it is also possible to simultaneously operate the system in the first image recording mode and in the second image recording mode such that at least one x-ray image (first image) and at least one second image (e.g. reflected light image, fluorescence image or luminescence image) can be generated at the same time.

In one variant, the beam splitter has a substrate made of a substrate material transparent to x-ray radiation and a plane substrate surface is coated with a dielectric alternating layer having a broadband effect. By way of example, said alternating layer can be designed in such a way that it has a high reflectivity (for example greater than 90%) in the visible wavelength range and in the adjacent UV wavelength range. The individual layers of the alternating layer system can be built up from fluoridic and/or oxidic materials with atoms with a relatively low atomic number, for example selected from the group SiO₂ and TiO₂. Such dielectric alternating layers can achieve the sought-after spectral broadband reflection effect without acting absorbent to an interfering extent for x-ray radiation. Here, the degree of absorption for x-ray radiation is substantially determined by the thickness and nature of the substrate material.

It may be expedient to coat an opposite plane substrate surface with a further coating, for example to counteract bending of a thin substrate due to layer tensions.

In another variant, the beam splitter has a substrate made of a substrate material transparent to x-ray radiation and a plane substrate surface is coated with a thin metal layer. By way of example, the layer thickness of the metal layer can be less than 10 micrometres, in particular less than 5 μm. Hence, the beam splitter be configured as a thin metallic mirror, wherein the metallic coating can consist of e.g. aluminium or silver and optionally comprise further layers, e.g. protective layers, serving the application. Since silver layers, for example, already have approximately the maximum reflectivity thereof for visible light at a layer thickness of approximately 100 nm and, at the same time, x-ray radiation is only attenuated to a very small extent in the case of layer thicknesses up to several micrometres, such metallic mirrors, which are relatively simple to produce, can be used as beam splitters in embodiments of the invention.

Within the meaning of this application, a substrate material is substantially transparent to x-ray radiation if the transmission of the substrate for x-ray radiation is more than 50%, in particular more than 60% or more than 70% or more than 80%.

If a plastic is selected as a substrate material, the absorption for x-ray radiation can be kept particularly low. It is also possible to select a substrate made of glass; this may be advantageous, for example for reasons of mechanical stability.

In general, it is considered to be advantageous to embody the substrate of the beam splitter to be as thin as possible in order to ensure the smallest possible absorption of the incident x-rays. Relatively thick substrates with a strength of several millimetres are used in conventional applications, particularly when using dielectric alternating layers or minor systems, in order to prevent bending of the substrate due to tensions generated by the dielectric alternating layers. However, such thick substrates can significantly attenuate the transmission of x-ray radiation such that an additional rear-side coating should preferably be applied in the case of dielectric beam splitters, said additional coating acting against the torsional stress and therefore enabling thinner substrates. This problem does not arise when using metallic mirrors since the stress torsion through the thin metal layer is negligible here. Therefore, metallic mirror layers can be applied to very thin substrates, e.g. microscope cover slips, with a strength of 1 mm or less, in particular less than 200 μm, and so the incident x-ray radiation experiences substantially no attenuation.

There are a number of variants in the case of embodiments with a beam splitter. In one variant, the beam splitter is fixedly installed such that it is permanently situated both in the optical path of the first imaging partial system and in the optical path of the second imaging partial system. This solution is simple from a constructional point of view and robust.

It is also possible to fasten the beam splitter to a movable carrier in order to selectively drive it into the beam splitter position (for examinations in the second image recording mode or both image recording modes) or drive it out of the beam path (for examinations in the first image recording mode). As a result of this, it is possible, for example, to spare the beam splitter at times, when no camera recordings are intended to be made over a relatively long period of time, or to increase the sensitivity for the first image recording mode since the x-ray radiation is incident directly on the detector without attenuation by the beam splitter if the beam splitter is swivelled out or driven out.

Particularly the imaging of living mice harbours problems, the reason for which is found in the nature of the object. Thus, what should be ensured in most image recording modes is that the object is stationary or unmoving, at least during the image recording time. In a preferred embodiment this can be achieved by virtue of the mice (or other small animals) being anaesthetized for the duration of carrying out the image recordings by means of an anaesthesia gas which is guided to the object in a controlled manner, preferably by way of a suitable mouthpiece. A development of the invention is characterized by a gas anaesthesia unit, which has an anaesthesia gas source, from which at least one gas-tight fluid line leads to a gas outlet arranged in the region of the object carrier. In the region of the gas outlet, the fluid line can merge into a mouthpiece opening to the outside, which is optionally adapted to an object to be recorded in such a way that the object at least partly fits into the mouthpiece. This allows reliable anaesthetization to be achieved.

Furthermore, as a result of the small body mass of mice, there quickly is the risk of these becoming hypothermic if they are mounted on supports without temperature control. Since the support for accommodating the object to be examined, i.e. the object carrier, should ensure a transmission of x-ray radiation which is as high as possible and, in particular, uniform, supports with thermostatic control which are based on resistance losses in current carriers are not considered to be well suited to this type of imaging. Therefore, some embodiments provide a temperature-control apparatus for controlling the temperature of an object, accommodated on the object carrier, by means of a (liquid or gaseous) fluid. To this end, the object carrier can have a carrier body made of a material transparent to x-ray radiation, with fluid channels for guiding a temperature-controllable fluid extending through the carrier body.

In such variants, a cavity is situated below the object bearing surface, to which cavity a temperature-controlled fluid medium, preferably air, can be supplied in a circulating manner, for example by way of a fan with a heating element disposed upstream or downstream thereof. In this case, the object bearing surface of the object carrier and at least the substantially horizontal boundary faces of the cavity preferably consist of a material that is as highly transmissive as possible to x-ray radiation, for example a plastic or glass fibre reinforced plastic (GRP), which combines high x-ray transmission with a high rigidity such that thin walls with no or few bracings can be realized.

Other objects, advantages and novel features of the present invention will become apparent from the following detailed description of one or more preferred embodiments when considered in conjunction with the accompanying drawings.

### BRIEF DESCRIPTION OF THE DRAWINGS

FIG. 1 shows a system for a multimodal imaging examination of objects in accordance with a first exemplary embodiment;

FIG. 2 shows a side view of some components in the region of the object carrier;

FIG. 3 shows a schematic plan view of the object carrier and adjacent components;

FIG. 4 shows part of an illumination apparatus, and

FIG. 5 shows a system for a multimodal imaging examination of objects in accordance with a second exemplary embodiment.

### DETAILED DESCRIPTION OF THE DRAWINGS

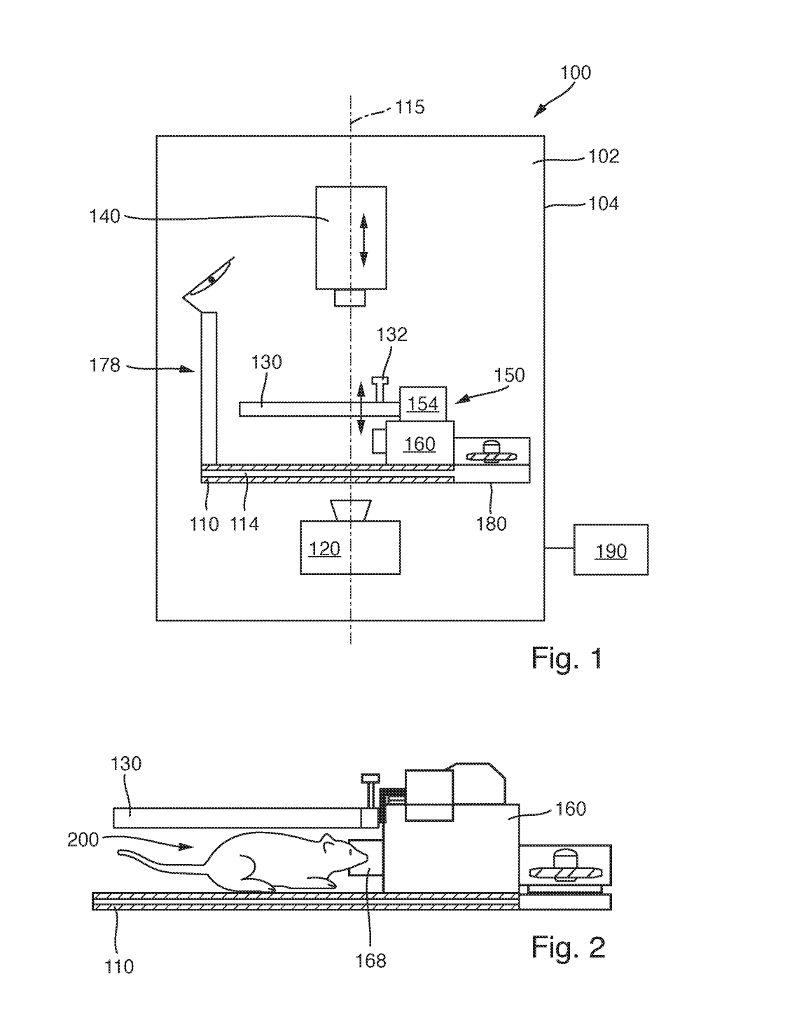

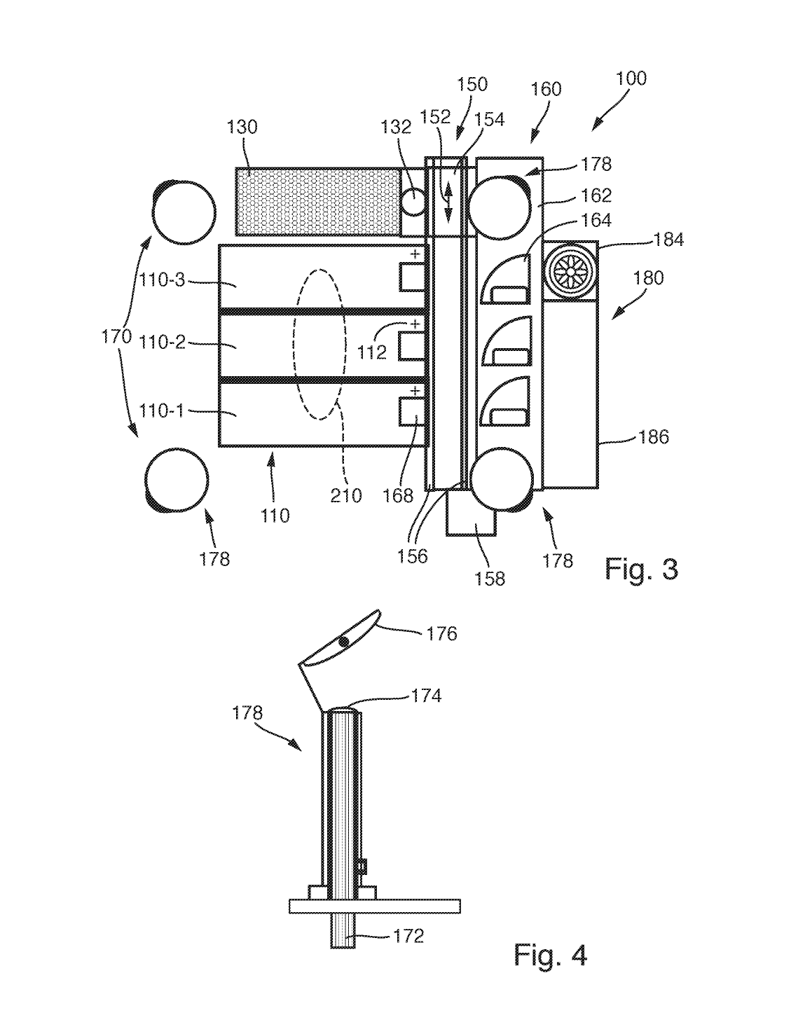

Schematic FIG. 1 shows a first exemplary embodiment of a system 100 for a multimodal imaging examination of objects. FIG. 2 shows a magnified side view of some components in the region of the object carrier; FIG. 3 shows a schematic plan view with an object carrier and adjacent components. The system 100 is suitable, inter alia, for in-vivo examinations of small animals, e.g. mice, rats or other organisms. The system allows examination of one and same object using different imaging methods, i.e. multimodal imaging. Images can be recorded in at least two different image recording modes, of which one can be an x-ray recording mode. Other possible image recording modes comprise image recording by means of fluorescence light, image recording by means of luminescence light and image recording by means of reflected light or light reflected by the object.

A flat, substantially plate-shaped object carrier 110 has on the horizontally aligned upper side thereof one or more object accommodating spaces 110-1, 110-2, 110-3, which are provided for accommodating an object 200 to be examined, for example a mouse. The object is placed or put onto the plane upper side of the object carrier 110 at the object accommodating space provided.

Situated at a distance below the object carrier is an x-ray radiation source 120, which is arranged in such a way that the x-rays radiated upward by the x-ray radiation source reach the object through the object carrier 110 and they are able to at least partly pass through said object. The x-ray radiation source can be operated in pulsed operation such that x-ray radiation is only radiated for short time intervals sufficient for x-ray image recordings. Variants with continuous operation (continuous wave) are also possible. Advantageously, the x-ray source comprises an optionally adjustable aperture which adapts the spatial spread in form and area to the area to be irradiated.

The object carrier 110 is constructed in such a way that x-ray radiation can pass to the object 200 from the x-ray radiation source 120 with as little impediment as possible. The carrying component of the object carrier is manufactured from a torsionally rigid plastic material in the exemplary case. Other materials with a low atomic number Z can also be used, for example aluminium-based and/or carbon-based materials such as carbon.

Situated at a distance above the object carrier 110 is a direct digital x-ray detector in the form of a fiat panel x-ray detector 130, which is aligned substantially horizontally in the shown image recording configuration and which is configured to convert the x-ray radiation which passed through the object and which was modified by the object into a digital image of the object. Here, the x-ray image recording mode is also referred to as first image recording mode; the recorded x-ray image is therefore also referred to as first image of the object.

In order to obtain a high image resolution, use is made of a planar (two-dimensional) flat panel x-ray detector with structured scintillators consisting of many individual scintillators delimited from one another, said scintillators being arranged in rows and columns extending perpendicular to one another. As a result, light scattering is avoided or reduced in comparison with a continuous, unstructured scintillator layer and the scintillation light is guided through the scintillator, mainly to the active pixel area (one or a few) assigned thereto and hence the image resolution is increased further since, as a result of this, there is no lateral spatial spread between point of incidence of the x-ray radiation and detection of the light signal.

Arranged at a distance above the flat panel x-ray detector 130 is a camera system 140, which is provided to record at least one second image of the same object 200 in at least one second image recording mode. In the exemplary case, this is a CCD camera with slow scan and backlit technology, which can be frozen down to −80° C. This technology offers a low background noise and highest quantum efficiency in a wavelength range, at least from approximately 440 nm to approximately 1100 nm, which is usable for fluorescence recordings, luminescence recordings and reflected light recordings. The optical axis of the optical unit of the camera system extends vertically. The camera system is displaceable in the vertical direction (z-direction) such that different image sections and magnifications are available in a simple manner. In the exemplary case, object dimensions in the range from 30 mm to 300 mm can be recorded in a substantially image-filling manner.

The x-ray radiation source 120, the object carrier 110, the flat panel x-ray detector 130 and the camera system 140 are arranged substantially along a common vertical axis 115 in the shown configuration.

As can be seen from the plan view in FIG. 3, the object carrier 110 provides a total of three directly adjacent, rectangular object accommodating spaces 110-1, 110-2 and 110-3, which each have a length-to-width ratio of approximately 3:1. Each one of the object accommodating spaces is dimensioned in such a way that a small experimental animal, for example a mouse, fits on the object accommodating space in the longitudinal direction. Overall, the object carrier has an approximately square base area, which is dimensioned in such a way that it lies completely in the region irradiatable by the x-ray source. Furthermore, the camera system can be adjusted in such a way that the whole base area of the object carrier lies in the image field of the camera system such that all three object accommodating spaces can be detected optically at the same time.

The aforementioned components are situated in the interior 102 of a substantially cuboid housing 104, which surrounds the interior in a light-tight manner. The plate-shaped wall parts of the housing consist of steel and are sufficiently thick for the housing not only to protect the interior from ambient light but also for it to act to shield the surroundings from x-ray radiation of the x-ray radiation source 120.

A display and operating unit 190 of the system is arranged outside of the housing 104 and it contains a computation unit (computer) for the central control of the system, by means of which the operation of the x-ray radiation source 120, of the object carrier 110, of the flat panel x-ray detector 130, of the camera system 140 and of further components of the system is controlled. Software modules for image evaluation are also active in the computer of the display and operating unit.

Further details of the system 100 can be identified from the magnified partial view in FIG. 2 and from the plan view in FIG. 3. The flat panel x-ray detector 130 is mounted in a movable manner such that it can be moved between different positions and fixed in the respective position. The flat panel x-ray detector 130 is accommodated in a holding apparatus which can be adjusted in the vertical direction by means of a height adjustment apparatus 132 such that the distance between the object carrier 110 and the flat panel x-ray detector 130 is continuously adjustable. Using this, it is possible, inter alia, to bring the flat panel x-ray detector close to the object 200 to be transilluminated by means of x-rays and to fix it there, and to adapt it to different object heights. As a result of this, the spatial resolution capability of the flat panel x-ray detector is used in an ideal way.

Furthermore, the flat panel x-ray detector 130 is fastened to a displacement apparatus 150 in such a way that it can be moved in a horizontal displacement direction 152 between different positions in a horizontal image recording plane extending perpendicular to the axis 115. To this end, the height-adjustable receptacle is fastened to a sled 154 which is guided linearly in the horizontal direction along straight guide rails 156. The displacement is controlled by way of an electric motor 158, which drives a horizontal threaded spindle, on which a spindle nut coupled to the sled 154 travels.

The system 100 comprises an illumination apparatus 170 which can be used for reflected light image recordings or for fluorescence excitation, either in an unmodified form or by fixing suitable optical filters in front thereof FIG. 4 schematically shows an assembly 178 of the illumination system, which an be denoted as a mirror projector assembly. The illumination apparatus 170 has four such identical assemblies and it is configured in such a way that light simultaneously falls as uniformly as possible and under a large solid angle from different directions onto the object to be imaged.

In the embodiment of FIG. 3, the light from a common light source (not shown here) is divided among four optical waveguides 172. The central, common light source can be equipped with excitation filters which are optionally insertable into the illumination beam path. The outlets of the optical waveguides 172 are distributed uniformly over the object carrier 110 or over the object accommodating spaces in a quadratic arrangement and radiate substantially upward in the vertical direction. The emerging light is collimated by means of a suitable optical unit (e.g. a collimation lens 174) and guided to a convex deflection mirror 176 substantially arranged in a 45°-adjustable manner, which widens the light cone and directs it onto the object obliquely from above. In another embodiment, this deflection can also be carried out by a plane mirror; the controlled widening of the light beam is then carried out by regulating the distance between collimation lens and optical waveguide outlet.

The object carrier 110 is heatable in order to prevent hypothermia of the (at least one) object. Since metallic heating spirals would be visible in the x-ray image in a bothersome manner, the heating is realized by way of an active flow of a temperature-controlled fluid medium in a cavity below the object. The temperature-controlled medium can be e.g. air or liquid. The system 100 of the exemplary embodiment comprises a temperature-control apparatus 180 for controlling the temperature of an object accommodated on the object carrier. With the aid of the temperature-control apparatus, the object carrier can be heated (or optionally cooled) from the inside, without the x-ray radiation passing through the object carrier being impeded. To this end, a channel system with fluid channels 114 is provided in the carrier body of the object carrier, through which a temperature-controllable fluid can be guided. In the exemplary case, the object carrier is heated from the inside by hot air. To this end, an electrical heating apparatus 184 is arranged outside of the object carrier, said heating apparatus being assigned a fan which, by way of an air suction channel 186, sucks in air from the outside. The electrically heated air is then guided through the fluid channels 114 in the interior of the object carrier and it thereby heats the latter. Since neither the fluid channels nor the fluid moving therein substantially influence the x-ray radiation, the x-ray image recordings are not impeded by the heating.

When examining living objects, such as e.g. mice, it is generally desirable for these not to be moving during the image recordings. In order to achieve this as sparingly as possible for the experimental animals, the system 100 is equipped with a gas anaesthesia unit 160, by means of which the animals can be anaesthetized with the aid of an anaesthetic gas. The gas is guided from an anaesthesia gas source arranged outside of the housing to the region of a valve block 162 by way of a gas-tight fluid line, with a switchable valve 164 being provided in said valve block for each one of the object accommodating spaces. From the valve, a line piece leads up to the region of a gas outlet in the region of the assigned object accommodating space. Situated in the region of the gas outlet there is a mouthpiece 168, the diameter of which is larger than that of the supply line, wherein this apparatus is adapted to an object to be accommodated in such a way that the object at least partly fits into the widened mouthpiece. In the exemplary case, the front end of the head of the mouse with nasal openings and mouth openings fits into the mouthpiece such that anaesthetic gas flows around the head from the front and the mouse can be anaesthetized reliably.

The x-ray radiation source and the flat panel x-ray detector are part of a first partial system for recording x-ray images in a first image recording mode. The camera system and the illumination system are part of a second partial system, by means of which e.g. a reflected light image, a fluorescence image and/or a luminescence image of the same object can be recorded in a second image recording mode. The remaining components are part of both partial systems.

The system 100 can be used for many different imaging examinations of one or more objects. By way of example, an x-ray image of the complete object or of part of the object can be recorded in a first image recording mode, which is also referred to as x-ray image recording mode, with the aid of the flat panel x-ray detector 130. To this end, the object is placed onto a suitable object accommodating space of the object carrier and immobilized when necessary. By way of example, if the object lies on the object accommodating space 110-1, the flat panel x-ray detector is displaced horizontally in the region above the object for the first image recording. Optionally, there is also a height adjustment in order to bring the flat panel x-ray detector as close as possible to the object (cf. FIG. 2). Then, the x-ray radiation source is switched on briefly in order to record a first digital image of the object, i.e. an x-ray image.

All that is required then for subsequently acquiring a second image with the aid of the camera system 140 is to displace the flat panel x-ray detector horizontally out of the region above the object by means of the displacement apparatus 150 such that the object can be recorded by means of the camera system. Then, for example, a reflected light image, a fluorescence image and/or a luminescence image of the object can be recorded in the second image recording mode. The illumination and the detection are adapted by fixing appropriate filters in front thereof, depending on the intended image type.

In another variant, a plurality of objects are placed onto the object carrier and immobilized simultaneously, for example three mice on the three object accommodating spaces 110-1, 110-2 and 110-3. Since the camera of the camera system can be adjusted in such a way that all three object accommodating spaces can be imaged simultaneously, the three objects can be recorded in a single first image recording. By contrast, the x-ray images are recorded sequentially or temporally in succession by virtue of the flat panel x-ray detector 130 being displaced in succession into the various image recording positions over the respective object accommodating spaces. The flat panel x-ray detector remains stationary in each case for the duration of the x-ray recording.

The x-ray image of an object is pieced together from a plurality of individual images in another image recording mode (stitching mode), said individual images in each case only containing a section of the overall object of interest. To this end, the flat panel x ray detector is successively displaced with the aid of the displacement apparatus 150 to mutually laterally offset image recording positions in the image recording plane thereof by way of a lateral displacement and it is fixed there in each case for the duration of an x-ray image recording. By way of example, it is possible to place a relatively large object 210, such as e.g. a rat, onto the object carrier in a manner substantially parallel to the displacement direction of the flat panel x-ray detector. The flat panel x-ray detector is then successively displaced in the horizontal direction to different image recording positions and kept there until an x-ray image is acquired. Here, the image recording positions are laterally offset to one another in such a way that the various offset images directly adjoin one another or partly overlap such that a gap-free overall image of the object 210 can be pieced together with the aid of the image evaluation software. In order to simplify later piecing together of the individual images, position markings 112, for example in the form of metal structures which generate a contrast in the x-ray image, are attached to the object carrier.

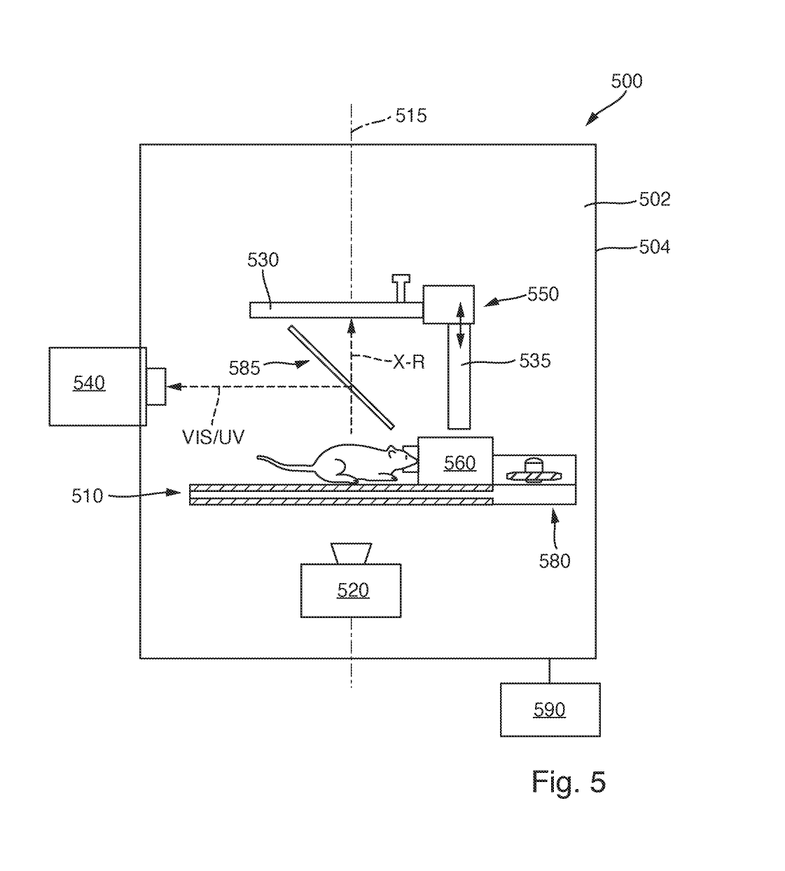

Schematic FIG. 5 shows a second exemplary embodiment of a system 500 for multimodal imaging and examining of objects. Components functionally equivalent or similar to those in the first exemplary embodiment are provided with the same reference sign increased by 400. The plate-shaped object carrier 510 with the gas anaesthesia unit 560 and the temperature-control apparatus 580 have exactly the same design as in the first exemplary embodiment; reference is made to the description there. A corresponding statement applies to the x-ray radiation source 520, which is arranged under the object carrier 510 and radiates x-ray radiation vertically upwards in general.

There are differences in the arrangement of the image-acquiring components of the first and second partial systems. Like in the first exemplary embodiment, the direct digital x-ray detector of the first partial system is a flat panel x-ray detector 530, but in this case it is mounted in a height-adjustable manner to a vertical guide apparatus 535. The sled and the horizontal guide rails of the displacement apparatus 550 are also arranged there. The vertical distance between the object carrier 510 and flat panel x-ray detector 530 is greater than in the first exemplary embodiment and dimensioned in such a way that a beam splitter 585 with a plane beam splitter face inclined at 45° in relation to the vertical axis 515 still fits between object carrier or object and flat panel x-ray detector. The two-dimensional extent of the beam splitter face is dimensioned in such a way that the perpendicular projection downward thereof largely covers the object accommodating spaces of the object carrier.

The beam splitter 585 of the exemplary embodiment is fixedly installed in the shown beam splitter position. The beam splitter consists substantially of a substrate in the form of a thin glass plate, the front side of which facing the object or the camera system being coated with a thin dielectric alternating layer which has a high reflectivity of more than 95% for visible wavelengths and wavelengths in the adjacent ultraviolet range (and near IR range). The spectral broadband reflection effect is achieved while having a low overall layer thickness (e.g. less than 1 μm) such that the alternating layer system is largely transmissive to x-ray radiation (transmission e.g. greater than 80% or greater than 90%). The thickness of the substrate material is selected in such a way that the beam splitter overall has a high transmissivity for x-ray radiation, wherein, for example, the overall absorption for x-ray radiation is no more than 10% or no more than 20%. By way of example, the thickness can be at 6 mm or less, in particular in the region of 0.5 mm to 3 mm.

The camera system 540 with a (horizontal) optical axis aligned perpendicular to the vertical axis 515 is arranged on a side wall of the housing 504 level with the beam splitter 585. Only the entrance optical unit is situated in the interior 502 of the housing; the active components of the camera system 540 are arranged outside of the interior. The beam path of the first partial system (for x-ray recordings) leads from the x-ray radiation source 520 through the object and the beam splitter 535 to the direct digital x-ray detector 530. The beam path of the second partial system (for image recordings of the camera system) is folded at the plane beam splitter face such that light emanating from the object (e.g. fluorescence light/luminescence light) and/or illumination radiation reflected and/or scattered by the object is reflected by the beam splitter face in the direction of the camera system.

An advantage of this embodiment consists of the fact that images in the first image recording mode (x-ray images) and images in the second image recording mode (e.g. reflected light images, fluorescence images) can be acquired at the same time or simultaneously. To this end, the x-ray radiation source and the camera system are operated at the same time. The x-ray radiation X-R is predominantly passed by the beam splitter 535 in the direction of the flat panel x-ray detector; the rest is absorbed such that practically no x-ray radiation reaches the camera system 540. By contrast the visible light and UV light (VIS/UV) emanating from the object is predominantly reflected in the direction of the camera system 540 such that reflected light images or fluorescence images can also be detected at the same time as the x-ray images.

Otherwise, this variant offers the same possibilities for use as the first embodiment.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A device for a multimodal imaging examination of an object, comprising:
an object carrier for accommodating at least one object to be examined;
an x-ray radiation source for emitting x-ray radiation;
a first partial system for generating a first image of the object in a first image recording mode, wherein the first partial system has a direct digital x-ray detector which is configured to convert x-ray radiation modified by the object into a digital image of the object; and
a second partial system for generating a second image of the object in a second image recording mode, wherein the second partial system has a camera system which is configured to convert radiation in the visible wavelength range or UV wavelength range emanating from an object into a digital image of the object.

2. The device according to claim 1, wherein the direct digital x-ray detector has a flat panel x-ray detector.

3. The device according to claim 1, wherein the direct digital x-ray detector has a pixel arrangement coated with a scintillator layer which converts x-ray radiation into visible light, wherein the scintillator layer is embodied as a laterally structured scintillator layer.

4. The device according to claim 1, wherein the direct digital x-ray detector is a direct x-ray detector which has a photoconductor sensitive to x-rays, said photoconductor generating charges which are proportional to the amount of incident radiation when x-ray photons are incident, without a scintillator being interposed.

5. The device according to claim 1, wherein the direct digital x-ray detector is movably mounted in such a way that the direct digital x-ray detector is movable between a defined first position and at least one defined second position.

6. The device according to claim 5, wherein:
the first position is an image recording position, suitable for recording an x-ray image, for the direct digital x-ray detector, in which the digital x-ray detector is arranged in a detection region of the camera system between the object carrier and the camera system, and
the direct digital x-ray detector is arranged outside of the detection region of the camera system in the second position in such a way that an object accommodated on the object carrier is detectable by the camera system.

7. The device according to claim 1, wherein the direct digital x-ray detector is fastened to a displacement apparatus in such a way that the direct digital x-ray detector is displaceable in an image recording plane from a first image recording position to at least one laterally offset second image recording position.

8. The device according to claim 1, wherein the x-ray radiation source, the object carrier, the direct digital x-ray detector and the camera system are arranged along a common axis in one configuration of the device.

9. The device according to claim 1, further comprising:
a beam splitter which is transparent to x-ray radiation and acts in a reflecting manner for visible light, wherein
the beam splitter is arranged or arrangeable in a beam splitter position between the object carrier and the direct digital x-ray detector in such a way that light in the visible wavelength range or UV wavelength range emanating from the object is reflectable via the beam splitter in the direction of the camera system and x-ray radiation modified by the object is transmitted in the direction of the direct digital x-ray detector.

10. The device according to claim 9, wherein the beam splitter has a substrate made of a substrate material transparent to x-ray radiation and a plane substrate surface is coated with a dielectric alternating layer, wherein a substrate surface lying opposite to the substrate surface preferably likewise carries a coating.

11. The device according to claim 9, wherein the beam splitter has a substrate made of a substrate material transparent to x-ray radiation and a plane substrate surface is coated with a thin metal layer, wherein the metal layer preferably contains aluminium or silver and/or a layer thickness of the metal layer is less than 10 μm and more than 100 nm.

12. The device according to claim 1, further comprising:
a gas anaesthesia unit, which has an anaesthesia gas source, from which at least one gas-tight fluid line leads to a gas outlet arranged in the region of the object carrier, wherein
a mouthpiece opening to the outside is arranged in the region of the gas outlet, which mouthpiece is adapted to an object to be recorded in such a way that the object at least partly fits into the mouthpiece.

13. The device according to claim 12, wherein the object carrier has a plurality of object accommodating spaces arranged next to one another, with a gas-light fluid line leading to each one of the object accommodating spaces.

14. The device according to claim 1, further comprising:
a temperature-control apparatus for controlling the temperature of an object, accommodated on the object carrier, by use of a fluid, wherein
the object carrier has a carrier body made of a material transparent to x-ray radiation, wherein fluid channels for guiding a temperature-controllable fluid extend through the carrier body.

15. The device according to claim 1, further comprising:
an illumination apparatus with at least one light source for illuminating an object accommodated by the object carrier from a side facing the camera system, wherein
the illumination apparatus is configured in such a way that the object is illuminable from different directions at the same time.

16. The device according to claim 1, further comprising:
a light-tight housing, which encloses a housing interior, wherein
at least the object carrier, the direct digital x-ray detector and an optical unit of the camera system are arranged in the housing interior, and
the x-ray radiation source is arranged in the housing interior as well.

17. A method for a multimodal imaging examination of an object, the method comprising the acts of:
accommodating the object on an object carrier;
irradiating the object with x-ray radiation from an x-ray radiation source;
generating a first image of the object in a first image recording mode by means of a direct digital x-ray detector which converts x-ray radiation modified by the object into a digital image of the object;
generating a second image of the object in a second image recording mode by means of a camera system which converts radiation in the visible wavelength range or UV wavelength range emanating from the object into a digital image of the object;
evaluating the first image and the second image.

18. The method according to claim 17, wherein the first image and the second image are generated at the same time.

19. The method according to claim 17, wherein the object is not moved for a change between the first image recording mode and the second image recording mode.

20. The method according to claim 17, wherein the direct digital x-ray detector is displaced in an image recording plane from a first image recording position to at least one laterally offset second image recording position, wherein:
(i) a plurality of laterally offset objects are recorded successively in succession by virtue of the direct digital x-ray detector initially being moved into an image recording position in relation to a first object and being used there for the recording of an image, and thereafter being moved into an image recording position in relation to a next object by way of a lateral displacement and being used there for the recording of an x-ray image, or
(ii) two or more laterally offset individual images of a single object are recorded in succession by virtue of a linear displacement of the direct digital x-ray detector in the image recording plane, which individual images are pieced together to form an overall image of the object.
